(19) United States
(12) Patent Application Publication
(10) Pub. No.: US 2004/0115170 A1
(43) Pub. Date: Jun. 17, 2004

(54) Oncolytic virus

(76) Inventors: Earl Garnet Brown, Nepean (CA); Jean Lutamyo Mbisa, Gloucester (CA); John Cameron Bell, Ottawa (CA); David Francis Stojdl, Ottawa (CA)

Correspondence Address: NIXON & VANDERHYE, PC, 1100 N GLEBE ROAD, 8TH FLOOR, ARLINGTON, VA, 22201-4714, US

(21) Appl. No.: 10/433,064

(22) PCT Filed: Nov. 30, 2001
(86) PCT No.: PCT/CA01/01703

(51) Int. Cl. A61K 48/00; C12N 15/86

(52) U.S. Cl. 424/93.2; 424/94.6; 435/456

(57) ABSTRACT

Methods of reducing the viability of a tumor cell, infecting a neoplasm in a mammal, utilizing certain non-naturally occuring viruses are disclosed. Viral reassortants, for example reovirus reassortants, and techniques for identifying PKR-sensitive viruses are also disclosed.

### SUMMARY OF THE INVENTION

This invention provides methods of reducing the viability of a tumor cell, infecting a neoplasm in a mammal with a virus, or treating a neoplasm in a mammal, comprising administering a non-naturally occurring virus wherein the virus is: a) a reovirus whose mu-2 protein has amino acid residues A, R, M, F, L, M, I, Q, I and S at positions 93, 150, 300, 302, 347, 372, 434, 458, 652 and 726, respectively; or b) a reassortant of two or more parent strains of a viral species selected from the family Reoviridae, or progeny thereof; or c) a virus other than a reovirus wherein the virus other than a reovirus is: i) capable of expressing a reovirus mu-2 protein having amino acid residues A, R, M, F, L, M, I, Q, I and S at positions 93, 150, 300, 302, 34.7, 372, 434, 458, 652 and 726, respectively, and ii) is a DNA virus, a positive-sense RNA virus, or a negative-sense RNA virus selected from the group consisting of Orthomyxoviridae, Rhabdoviridae and Paramyxoviridae. This invention father provides the use of such non-naturally occurring virus in the manufacture of a medicament for reducing the viability of a tumor cell, infecting a neoplasm in a mammal, or treating a neoplasm in a mammal.

This invention provides a method of identifying a PKR sensitive virus comprising: a) dividing a sample of a virus to be tested into a first portion and second portion; b) contacting PKR +/+ cells with the first portion and contacting PKR −/− cells with the second portion, under conditions permitting growth of the virus in PKR −/− cells; c) determining the rate of growth of the virus in the PKR +/+ cells and in the PKR −/− cells; and d) comparing the growth rates from step c), wherein a higher rate of growth in the PKR −/− cells than in the PKR +/+ cells identifies the virus as PKR sensitive. Such PKR sensitive viruses identified in accordance with this invention are useful for reducing the viability of a tumor cell, infecting a neoplasm in a mammal, or treating a neoplasm in a mammal.

### DESCRIPTION OF THE FIGURES

FIG. 1: Virus yield of reovirus strains T1L and T3D in PKR −/− vs. PKR +/+ murine embryo fibroblasts.

FIG. 2: Immuno-blot of PKR in MEF Infected with Reo T1L and T3D.

FIG. 3: Lungs of mice with ct26 tumors after treatment with reovirus strains.

T1L, T3D, EB96, EB108 and EB146 relative to untreated control lung. The lungs from 2 mice are shown for each treatment

FIG. 4: The weight of BALB-C mouse lungs relative to the presence of CT26 tumors and reovirus treatment.

FIG. 5: Histological sections stained with hematoxylin and eosin showing lung lobes of mice with ct26 tumors after treatment with reovirus strains. T1L, T3D, EB96, EB108 and EB146 relative to untreated control lung.

### DETAILED DESCRIPTION OF THE INVENTION

Throughout this application amino acids are generally identified using the standard one-letter abbreviation, but can also be identified by name or standard three-letter abbreviations.

T3D, T1L, T3A and T2J are standard abbreviations for reovirus strains T3 Dearing, T1 Lang, T3 Abney, and T2 Jones, respectively. The above-listed names of strains and their respective abbreviations are used interchangeably.

As used herein “phenotype” refers to the sequence of the expressed proteins of a virus. In the case of reoviruses the expressed proteins are the gene products of the L1, L2, L3, M1, M2, M3, S1, S2, S3 and S4 genes. Thus, if the amino acid sequences of the products of these genes are the same in two different reoviral strains they are said to have the same phenotype.

As used herein “genotype” refers to the nucleotide sequence of the coding region of a virus. Thus, for example, if the nucleotide sequences of the L1, L2, L3, M1, M2, M3, S1, S2, S3 and S4 genes of two reoviruses are the same in two different reoviral strains they are said to have the same genotype.

The term “PFU” stands for plaque forming units and is a quantitative measure of live virus particles.

Examples of the anti-neoplastic and anti-tumor methods and use of this invention as described above, include those utilizing a reovirus whose mu-2 protein has amino acid residues A, R, M, F, L, M, I, Q, I and S at positions 93, 150, 300, 302, 347, 372, 434, 458, 652 and 726, respectively. In a more specific embodiment the reeoviral mu-2 protein has the amino acid sequence of the mu-2 protein of reovirus strain T3 Dearing, for example when the mu-2 protein is expressed by a gene having the nucleic acid sequence of the M1 gene of reovirus strain T3 Dearing. In a more specific embodiment the reovirus has the same genotype as a reovirus strain selected from the group consisting of eb86, eb129, eb88, eb13, and eb145. In a more specific embodiment the reovirus has a M1 gene whose sequence is the same as the M1 gene of reovirus strain T3 Dearing and an L3 gene whose sequence is the same as the L3 gene of reovirus strain T1 Lang, for example the virus can have the same genotype as a reovirus strain selected from the group consisting of eb28, eb31, eb97, eb123 and g16. In a still more specific embodiment the reovirus has a M1 gene whose sequence is the same as the M1 gene of reovirus strain T3 Dearing and an L3 gene, L1 gene, and S2 gene whose sequences are the same as the corresponding genes of reovirus strain T1 Lang, for example reoviruses having the same genotype as a reovirus strain selected from eb96, eb146 and eb108. In an even more specific embodiment the reovirus has a M1 gene whose sequence is the same as the M1 gene of reovirus strain T3 Dearing and an L3 gene, L1 gene, S2 gene and S4 gene whose sequences are the same as the corresponding genes of reovirus strain T1 Lang, for example reoviruses having the same genotype as reovirus strain eb96.

Other examples of the anti-neoplastic and anti-tumor methods and use of this invention as described above, include those utilizing a virus that is a reassortant of two or more parent strains of a viral species selected from the family Reoviridae, or progeny thereof. For example, reassortants can be made of two, three or four of the reovirus strains T3 Dearing, T1 Lang, T3 Abney, and T2 Jones. In a more specific embodiment the reassortants are generated from parent strains T3 Dearing and T1 Lang. Examples of such strains include eb118, eb73.1, h17, h15, eb39, and h60 as well as the other stains shown in Tables 1 and 2.

Other examples of the anti-neoplastic and anti-tumor methods and use of this invention as described above, include those utilizing a virus other than a reovirus that is: i) capable of expressing a reovirus mu-2 protein having amino acid residues A, R, M, F, L, M, I, Q, I and S at positions 93, 150, 300, 302, 347, 372, 434, 458, 652 and 726, respectively, and ii) is a DNA virus, a positive-sense RNA virus, or a negative-sense RNA virus selected from the group consisting of the families Orthomyxoviridae, Rhabdoviridae and Paramyxoviridae. Examples of suitable DNA viruses include a Herpesvirus, Adenovirus, Parvovirus, Papovavirus, Iridovirus, Hepadenavirus, Poxvirus, mumps virus, human parainfluenza virus, measles virus or rubella virus. Examples of suitable a positive-sense RNA viruses include a Togavirus, Flavivirus, Picornavirus, or Coronavirus. Examples of suitable negative-sense RNA virus selected from the group consisting of Orthomyxoviridae, Rhabdoviridae and Paramyxoviridae include an influenza virus or a vesicular stomatitis virus.

In accordance with the method of identifying a PKR sensitive virus of this invention as described above, any PKR +/+ and −/− cells can be used, and the rate of growth of the virus is determined by any standard technique for monitoring viral growth including those that measure the number of virus particles directly or the quantity of viral proteins. In a specific embodiment the PKR cells are mouse embryo fibroblasts. In another specific embodiment the rate of growth of the virus is determined by a technique selected from the group consisting of plaque titer assay, antibody assay, and Western blot. Each of these techniques is exemplified below. Preferably the growth rate of the virus in PKR −/− cells is at least ten times higher than the growth rate in PKR +/+ cells.

In all of the anti-neoplastic and anti-tumor methods and use of this invention as described above, the virus can be a replication competent virus and/or a clonal virus. The virus can be administered by any conventional route, including but not limited to intranasally, intratracheally, intravenously, intraperitoneally or intratumorally. In accordance with the method or use of reducing the viability of a tumor cell described above, the virus can be administered to the tumor cell either in vivo or ex vivo. When the virus is administered to a mammal, the mammal can be either a human or a non-human mammal such as a mouse, sheep, cow, pig, dog or rabbit. While the optimal dose is expected to differ somewhat from patient to patient and can readily be determined by a skilled clinician, a dosage of from 3×10⁷ to 3×10⁹ PFU/kg is typical.

The viruses utilized in accordance with this invention can be produced by any conventional means, including reassortrnent among two or more parent virus strains or the use of standard recombinant genetic techniques. Once produced, such viruses can be reproduced by culturing in cells to produce progeny. The construction of reassortants of viruses is well known and is described, for example in Brown, et al., “The L2 Gene of Reovirus Serotype 3 Controls the Capacity to Interfere, Accumulate Deletions and Establish Persistent Infection” in Double-Stranded RNA Viruses, Compans, et al. eds. Elsevier (1983). For example, reassortants can be made of two, three or four of the reovirus strains T3 Dearing, T1 Lang, T3 Abney, and T2 Jones. Reassortants of T3 Dearing and T1 Lang are described in Example 2. Preferably the virus is replication competent and/or a clonal virus.

This invention will be better understood by reference to the following examples, which illustrate but are not intended to limit the invention described herein.

### Experiments

Experiment 1: Growth of Reovirus Strains T1L and T3D in PKR Knock-Out and Wild Type Fibroblast Cells

Viral Growth

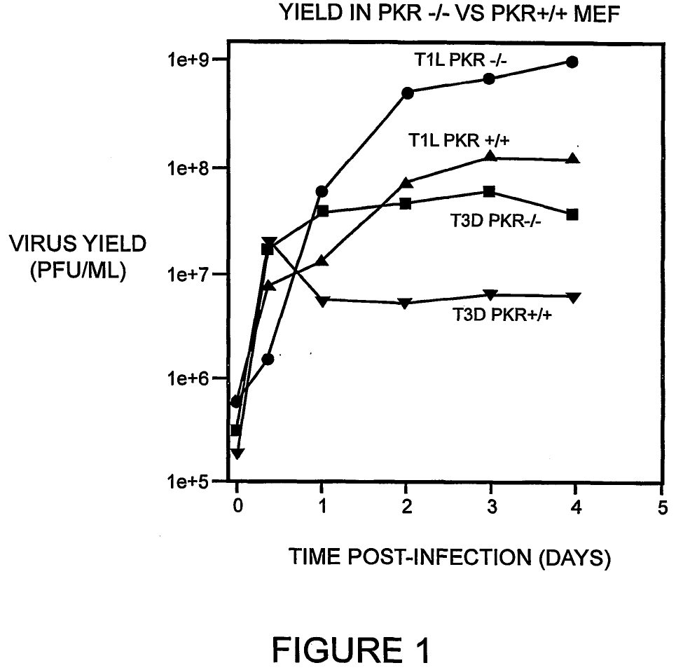

The effect of PKR on reovirus infection was examined using PKR knock-out (PKR −/−) murine embryo fibroblasts (MEF). Both reovirus T1L and T3D grow to several fold higher titre in PKR −/− relative to PKR +/+ MEF, as measured by plaque assay. (FIG. 1) This was associated with a higher percentage of antigen positive cells detected by fluorescent antibody staining described below. Consistent with this, infection of PKR −/− MEF resulted in several fold greater amounts of viral protein as assayed by western blot described below. Although both T1L and T3D grew to higher titres in cells lacking the PKR gene T1L virus grew to higher titres than T3D in either PKR −/− or PKR +/+ cells. (FIG. 1)

### Indirect Immunostaining

Cells were grown on glass coverslips in 35 mm diameter dishes and were infected with reovirus T1L or T3D at a multiplicity of infection (moi) of 10. After 48 hours incubation the cells were rinsed in PBS and fixed in prechilled acetone for 5 min. After rinsing in PBS (3×5 min), 100 μl of an appropriate dilution of type-specific rabbit antivirus antisera was applied and incubated at room temperature for 30 min. The coverslips were then rinsed in PBS (3×5 min) and treated with the appropriate dilution of Cy3-conjugated donkey anti-rabbit antibody (Jackson ImmunoResearch Laboratories, Inc.) as the secondary antibody. After another 30 min incubation period at room temperature the coverslips were rinsed in PBS (3×5 min) and mounted on glass slides in Gel/Mount (Biomeda Corp). All antibody dilutions were done in PBS/3% BSA.

The samples were examined with a Zeiss microscope equipped with epifluorescence and a 40×1.40 NA PlanApo objective. The images were collected using Image One Metamorph software and a Hamamatsu chilled charge-coupled digital camera (model C5985). Configuration of the digital images was done using Corel Presentations software.

Immunoblotting

Monolayer cultures of MEF were infected at a moi=10 with T1L or T3D virus as described above. At various times the culture medium was removed and the cells were rinsed with PBS before solubilizing in 1 ml of sample buffer (62.5 mM Tris-HCl pH6.8, 10% glycerol, 2% SDS, 0.05% bromophenol blue and 5% 2-mercaptoethanol)(Laemmli). Aliquots of 25 ul volume were subjected to SDS PAGE and transblotted onto an Immobilon P membrane (Millipore) at 25V overnight at 4° C. The dried membrane was blocked with 5% (w/v) skim milk powder in PBS for 1 hr at RT. This was followed by the addition of type specific rabbit anti-reovirus immune serum as the primary antibody in fresh blocking solution and incubation for 2 hr at 4° C. The membrane was then washed three times in PBS and once in TBS (100 mM Tris Hcl pH 7.4, 0.9% NaCl) to remove phosphate and incubated in 5% milk in TBS containing 1 ug/ml protein A conjugated to alkaline phoshatase obtained from Sigma Chemicals (Oakville, Ont) Finally the membrane was washed 4× in TBS before reaction with chromogenic substrate, nitro blue tetrazolium (NBT) (33 ug/ml) plus 5-bromo-4-chloro-3-indolyl phoshate (BCIP) (3.3 ul/ml), in alkaline phosphatase buffer (100 mM NaCl, 5 mM MgCl2 and 100 mM Tris-HCl pH9.5). The reaction was stopped with PBS containing 20 mM EDTA.

Experiment 2: Reassortants Between Reovirus Strains T1L and T1D

Production of Genetic reassortants between Reovirus Serotype 1 Lang strain and Serotype 3 Dearing strain.

Mouse L929 cells were coinfected with Reovirus Serotype 1 Lang strain (T1L) and Serotype 3 Dearing strain (T3D) at a multiplicity of infection of 5 each. Virus was harvested 24 hr post infection by 3 cycles of freezing and thawing before progeny viruses were isolated by 2 cycles of plaque isolation in L929 monolayers. Since each of the corresponding genome segments of T1L and T3D is distinguishable by electrophoretic mobility the genetic composition of each virus was determined by polyacrylamide gel electrophoresis of the segmented double stranded RNA (dsRNA) genome where the mobility of each segment is compared to the parental strains. Gels prepared as described by Laemmli contained 10% polyacrylamide and 0.27% methylene bis-acrylamide. Double-stranded RNA was obtained from L929 cells infected for 3 days and solubilised in buffer containing sodium dodecyl sulphate and was detected in gels stained with ethidium bromide as described previously (Zou S. and E. G. Brown. (1992) Identification of Sequence elements containing signals for replication and encapsidation of the reovirus M1 genome segment. Virology 186:377-88. The use of this panel of reassortants was first described by E. G. Brown, M. L. Nibert and B. N. Fields (1983) The L2 gene of reovirus serotype 3 controls the capacity to interfere, accumulate deletions and establish persistent infection in Double-Stranded RNA Viruses. R. W. Compans and D. H. L. Bishop eds. Elsevier Science Publishing Co.

Growth of Reovirus

T1L, T3D and virus stocks from the reassortment procedure described above were prepared in L929 cells grown in Earl's Minimal Essential Medium (MEM) supplemented with 5% fetal bovine serum and penicillin to 100 units/ml and streptomycin to 100 ug/ml until cytopathic effect was complete. Cells and culture supernatant were subjected to 3 cycles of freezing and thawing before titration by plaque assay.

Yields in Mouse Embryo Fibroblasts

Wild type PKR +/+ cells were obtained from Balb-C mice and PKR −/− cells were obtained from PKR knockout mice. Cell cultures were produced using 15-17 days embryos that had been disaggregated by mincing and trypsin treatment. Cell monolayers were grown in 35 mm plastic dishes in MEM supplemented with 10% FBS and P/S at 37 C in a 5% CO₂ atmosphere. Cells were infected with titrated T1L, T3D or reassortant reovirus at a multiplicity of infection (moi) of 10 by adsorption of stock virus for 0.5 hr with agitation at 15 minute intervals. Unadsorbed virus was removed by 3 washes with 2 ml of warm PBS each before the addition of 3 ml of MEM supplemented with 5% fetal bovine serum and penicillin to 100 units/ml and streptomycin to 100 ug/ml. The yield of T1L and T3D was assayed at time points over a 4 day period and is shown in FIG. 1. Comparison of yields of virus from MEF cells infected with reassortant reovirus was done after 3 days incubation by plaque assay of duplicate cultures. The results are shown below in Table 1 (PKR −/−) and Table 2 (PKR +/+).

Plaque Assay of Reovirus in L929 Cells

Monolayer cultures of L929 cells were decanted of medium and infected in duplicate with 0.1 ml volumes of serially diluted virus in PBS. Virus was adsorbed for 0.5 hr before the application of 3 ml of MEM supplemented with 1% agar, 5% FBS and P/S. Cultures were incubated at 37 C and supplementary overlays of 2 ml aliquots of the same medium was added 3 and 6 days post infection. After 8 days of infection the monolayers were stained for 24 hr with 2 ml of the same overlay solution supplemented with neutral red (0.01% weight/volume) to observe plaques.

Discussion

The genetic basis for the increased ability of T1L to grow in each cell type was determined using T1L×T3D reassortants. The difference in yield in wild type MEF (PKR +/+) segregated primarily with the M1 gene whereas the difference in yield in PKR −/− MEF was associated with the L1, L3, M3 and S2 genes and did not involve the M1 gene. The comparison of the genetic basis for replication in PKR +/+ relative to PKR −/− MEF cells indicates that the ability of the PKR gene to inhibit reovirus infection is dependent on the properties of the M1 gene. Furthermore the extent of replication and thus exploitation of PKR −/− cells is dependent on the nature of the L1, L3, M3 and S2 genes. Thus the reassortant viruses with the greatest differential ability to replicate in PKR −/− relative to PKR +/+ cells possess the T3D M1 gene and the viruses with the greatest ability to replicate in PKR −/− cells (characteristic of many tumor cells) possess the L1, L3, M3 and S2 genes of T1L. Such viruses are restricted in replication of PKR +/+ cells but replicate to a greater extent than either T1L or T3D in PKR −/− cells and are embodied in the properties of the reassortants eb96 and eb108. Statistical analyses of the experimental results are shown in Tables 1, 2 and 3.

The amino acid sequences of the T1L and T3D mu2 proteins are shown in Table 4. Each protein is 736 amino acids long and they differ at 10 aa positions. The observed difference in sensitivity to PKR seen as an ability to replicate in PKR +/+ relative to PKR −/− MEF cells is attributed to the difference in amino acid sequence between these proteins and thus M1 proteins of reoviruses with these amino acid changes or other substitutions at these positions are addressed herein. The mu2 protein is encoded by the M1 gene. The nucleotide sequences of the T1L and T3D M1 gene are shown in Table 5. Each genome segment is 2304 nucleotides long and they differ at 51 nucleotide positions.

TABLE 1
PKR −/−

| VIRUS | TITRE | L1 | L2 | L3 | M1 | M2 | M3 | S1 | S2 | S3 | S4 | RANK |
|---|---|---|---|---|---|---|---|---|---|---|---|---|
| eb146 | 7.00E+08 | L | L | L | D | L | L | L | L | L | D | 1 |
| eb28 | 5.80E+08 | D | D | L | D | D | D | D | L | D | D | 2 |
| eb108 | 4.70E+08 | L | D | L | D | L | L | L | L | D | D | 3 |
| eb118 | 4.50E+08 | D | D | L | L | D | D | D | D | L | L | 4 |
| T1L | 4.30E+08 | L | L | L | L | L | L | L | L | L | L | 5 |
| eb73.1 | 3.50E+08 | L | D | L | L | D | D | D | D | D | D | 6 |
| eb31 | 3.20E+08 | L | L | L | D | L | L | L | D | D | L | 7 |
| h17 | 3.00E+08 | D | D | L | L | D | D | L | D | D | L | 8 |
| H15 | 2.80E+08 | L | D | D | L | D | D | D | D | D | L | 9 |
| eb39 | 2.60E+08 | L | D | D | L | D | D | D | D | D | D | 10 |
| eb96 | 1.80E+08 | L | D | L | D | L | L | L | L | D | L | 11 |
| eb97 | 1.40E+08 | D | D | L | D | D | D | D | D | D | L | 12 |
| h60 | 1.30E+08 | D | D | L | L | D | D | D | D | D | L | 13 |
| T3D | 1.20E+08 | D | D | D | D | D | D | D | D | D | D | 14 |
| eb123 | 9.50E+07 | D | D | L | D | D | D | D | D | L | D | 15 |
| g16 | 9.30E+07 | L | L | L | D | L | L | L | D | L | L | 16 |
| eb86 | 8.50E+07 | L | D | D | D | D | L | D | D | D | L | 17 |
| eb129 | 6.30E+07 | D | D | D | D | D | L | D | L | L | D | 18 |
| eb88 | 6.00E+07 | D | D | D | D | L | D | D | D | D | D | 19 |
| eb13 | 5.30E+07 | D | D | D | D | D | D | D | D | D | L | 20 |
| eb145 | 1.30E+07 | D | D | D | D | D | L | L | D | D | D | 21 |
| | t-test | 0.045 | 0.19 | 0.019 | 0.024 | 0.25 | 0.75 | 0.57 | 0.087 | 0.62 | 0.76 | |
| | M-W test | 0.085 | 0.19 | 0.007 | 0.109 | 0.28 | 1 | 0.26 | 0.047 | 0.61 | 0.97 | |

TABLE 2
PKR +/+ (wild type)

| VIRUS | TITRE | L1 | L2 | L3 | M1 | M2 | M3 | S1 | S2 | S3 | S4 | RANK |
|---|---|---|---|---|---|---|---|---|---|---|---|---|
| h60 | 3.96E+08 | D | D | L | L | D | D | D | D | D | L | 1 |
| eb39 | 2.35E+08 | L | D | D | L | D | D | D | D | D | D | 2 |
| H15 | 1.78E+08 | L | D | D | L | D | D | D | D | D | L | 3 |
| eb118 | 1.76E+08 | D | D | L | L | D | D | D | D | L | L | 4 |
| eb146 | 1.68E+08 | L | L | L | D | L | L | L | L | L | D | 5 |
| T1L | 1.50E+08 | L | L | L | L | L | L | L | L | L | L | 6 |
| h17 | 1.46E+08 | D | D | L | L | D | D | L | D | D | L | 7 |
| eb28 | 1.30E+08 | D | D | L | D | D | D | D | L | D | D | 8 |
| eb73.1 | 1.23E+08 | L | D | L | L | D | D | D | D | D | D | 9 |
| eb31 | 5.20E+07 | L | L | L | D | L | L | L | D | D | L | 10 |
| eb123 | 4.88E+07 | D | D | L | D | D | D | D | D | L | D | 11 |
| g16 | 4.03E+07 | L | L | L | D | L | L | L | D | L | L | 12 |
| eb129 | 3.78E+07 | D | D | D | D | D | L | D | L | L | D | 13 |
| eb97 | 2.35E+07 | D | D | L | D | D | D | D | D | D | L | 14 |
| eb96 | 2.20E+07 | L | D | L | D | L | L | L | L | D | L | 15 |
| eb108 | 1.33E+07 | L | D | L | D | L | L | L | L | D | D | 16 |
| T3D | 1.20E+07 | D | D | D | D | D | D | D | D | D | D | 17 |
| eb13 | 7.50E+06 | D | D | D | D | D | D | D | D | D | L | 18 |
| eb86 | 6.40E+06 | L | D | D | D | D | L | D | D | D | L | 19 |
| eb88 | 6.00E+06 | D | D | D | D | L | D | D | D | D | D | 20 |
| eb145 | 2.25E+06 | D | D | D | D | D | L | L | D | D | D | 21 |
| | t-test | 0.39 | 0.15 | 0.056 | 0.0001 | 0.68 | 0.2 | 0.76 | 0.56 | 0.1 | 0.48 | |
| | M-W test | 0.4 | 0.35 | 0.07 | 0.0009 | 0.63 | 0.21 | 0.8 | 0.85 | 0.24 | 0.42 | |

In Tables 1 and 2, parental origin of genome segments is indicated by L (T1L) or D (T3D). Statistical significance was determined using the t-test and the Mann-Whitney (MW) test.

TABLE 3
SUSCEPTIBILITY TO PKR SEGREGATES WITH THE M1
GENE

| | Single gene regression | | Stepwise regression | |
|---|---|---|---|---|
| | (R² %) | | (R² %) | |
| Gene | PKR+/+ | PKR−/− | PKR+/+ | PKR−/− |
| L1 | 0 | 19 (P = .048) | 0 | L3 + L1 |
| | | | | 48 (P = .003) |
| L3 | 23.8 | 36 (P = .004) | M1 + L3 67.0 | 36 (P = .004) |
| | (P = .025) | | (P < .001) | |
| M1 | 51.6 | 0 | 51.6 | L3 + L1 + M1 |
| | (P < .001) | | (P = <.001) | 56 (P = .0025) |
| S2 | 0 | 16 (P = .073) | 0 | L3 + L1 + M3 + S2 |
| | | | | 63.4 (P < .001) |

TABLE 4

Alignment of T1L (GenBank Accession No. CAA42570.1) and T3D
(GenBank Accession No. AAA47256.1) mu2 proteins. These amino acid sequences
were deduced from cDNA. Each protein is 736 nucleotides long and differs at 10 aa
positions.

| | | | |
|---|---|---|---|
| T1L | 1 | MAYIAVPAVVDSRSSEAIGLLESFGVDAGADANDVSYQDHDYVLDQLQYMLDGYEAGDVI | 60 |

| | |
|---|---|
| Consensus | MAYIAVPAVVDSRSSEAIGLLESFGVDAGADANDVSYQDHDYVLDQLQYMLDGYEAGDVI |

| | | | |
|---|---|---|---|
| T3D | 1 | MAYIAVPAVVDSRSSEAIGLLESFGVDAGADANDVSYQDHDYVLDQLQYMLDGYEAGDVI | 60 |
| T1L | 61 | DALVHKNWLHHSVYCLLPPKSQLLEYWKSNPSVIPDNVDRRLRKRLMLKKDLRKDDEYNQ | 120 |

| | |
|---|---|
| Consensus | DALVHKNWLHHSVYCLLPPKSQLLEYWKSNPSIPDNVDRRLRKRLMLKKDLRKDDEYNQ |

| | | | |
|---|---|---|---|
| T3D | 61 | DALVHKNWLHHSVYCLLPPKSQLLEYWKSNPSAIPDNVDRRLRKRLMLKKDLRKDDEYNQ | 120 |
| T1L | 121 | LARAFKISDVYAPLISSTTSPMTMIQNLNQGEIVYTTTDRVIGARILLYAPRKYYASTLS | 180 |

| | |
|---|---|
| Consensus | LARAFKISDVYAPLISSTTSPMTMIQNLNGEIVYTTTDRVIGARILLYAPRXYYASTLS |

| | | | |
|---|---|---|---|
| T3D | 121 | LARAFKISDVYAPLISSTTSPMTMIQNLNRGEIVYTTTDRVIGARILLYAPRKYYASTLS | 180 |
| T1L | 181 | FTMTKCIIPFGKEVGRVPHSRFNVGTFPSIATPKCFVMSGVDIESIPNEFIKLFYQRVKS | 240 |

| | |
|---|---|
| Consensus | FTMTKCIIPFGKEVGRVPHSRFNVGTFPSIATPKCFVMSGVDIESIPNEFIKLFYQRVKS |

| | | | |
|---|---|---|---|
| T3D | 181 | FTMTKCIIPFGKEVGRVPHSRFNVGTFPSIATPKCFVMSGVDIESIPNEFIKLFYQRVKS | 240 |
| T1L | 241 | VHANILNDISPQIVSDMINRKRLRVHTPSDRRAAQLMHLPYHVKRGASHVDVYKVDVVDV | 300 |

| | |
|---|---|
| Consensus | VHANILNDISPQIVSDMINRKRLRVHTPSDRRAAQLMHLPYHVKRGASHVDVYKVDVVD |

| | | | |
|---|---|---|---|
| T3D | 241 | VHANILNDISPQIVSDMINRKRLRVHTPSDRRAAQLMHLPYHVKRGASHVDVYKVDVVDM | 300 |
| T1L | 301 | LLEVVDVADGLRNVSRKLTMHTVPVCILEMLGIEIADYCIRQEDGMFTDWFLLLTMLSDG | 360 |

| | |
|---|---|
| Consensus | L EVVDVADGLRNVSRKLTMHTVPVCILEMLGIEIADYCIRQEDGMTDWFLLLTMLSDG |

| | | | |
|---|---|---|---|
| T3D | 301 | LFEVVDVADGLRNVSRKLTMHTVPVCILEMLGIEIADYCIRQEDGMLTDWFLLLTMLSDG | 360 |
| T1L | 361 | LTDRRTHCQYLINPSSVPPDVILNISITGFINRHTIDVMPDIYDFVKPIGAVLPKGSFKS | 420 |

| | |
|---|---|
| consensus | LTDRRTHCQYLNPSSVPPDVILNISITGFINRHTIDVMPDIYDFVKPIGAVLPKGSFKS |

| | | | |
|---|---|---|---|
| T3D | 361 | LTDRRTHCQYLMNPSSVPPDVILNISITGFINRHTIDVMPDIYDFVKPIGAVLPKGSFKS | 420 |
| T1L | 421 | TIMRVLDSISILGVQIMPRAHVVDSDEVGEQMEPTFEHAVMEIYKGIAGVDSLDDLIKWV | 480 |

| | |
|---|---|
| Consensus | TIMRVLDSISILG QIMPRAHVVDSDEVGEQMEPTFEAVMBIYKGIAGVDSLDDLIKWV |

| | | | |
|---|---|---|---|
| T3D | 421 | TIMRVLDSISILGIQIMPRAHVVDSDEVGEQMEPTFEQAVMEIYKGIAGVDSLDDLIKWV | 480 |
| T1L | 481 | LNSDLIPHDDRLGQLFQAFLPLAKDLLAPMARKFYDNSMSEGRLLTFAHADSELLNANYF | 540 |

| | |
|---|---|
| Consensus | LNSDLIPHDDRLGQLFQAFLPLAKDLLAPMARKFYDNSMSEGRLLTFAHADSELLNANYF |

| | | | |
|---|---|---|---|
| T3D | 481 | LNSDLIPHDDRLGQLFQAFLPLAKDLLAPMARKFYDNSMSEGRLLTFAHADSELLNANYF | 540 |
| T1L | 541 | GHLLRLKIPYITEVNLMIRKNREGGELFQLVLSYLYKMYATSAQPKWFGSLLRLLICPWL | 600 |

| | |
|---|---|
| Consensus | GHLLRLKIPYITEVNLMIRKNREGGELFQLVLSYLYKMYATSAQPKWFGSLLRLLICPWL |

| | | | |
|---|---|---|---|
| T3D | 541 | GHLLRLKIPYITEVNLMIRKNREGGELFQLVLSYLYKMYATSAQPKWFGSLLRLLICPWL | 600 |
| T1L | 601 | HMEKLIGEADPASTSAEIGWHIPREQLMQDGWCGCEDGFIPYVSIRAPRLVMEELMEKNW | 660 |

| | |
|---|---|
| consensus | HMEKLIGEADPASTSAEIGWHIPREQLMQDGWCGCEDGFIPYVSIRAPPLVEELMEKNW |

| | | | |
|---|---|---|---|
| T3D | 601 | HMEKLIGEADPASTSAEIGWHIPREQLMQDGWCGCEDGFIPYVSIRAPRLVIEELMEKNW | 660 |
| T1L | 661 | GQYHAQVIVTDQLVVGEPRRVSAKAVIKGNHLPVKLVSRFACFTLTAKYEMRLSCGHSTG | 720 |

| | |
|---|---|
| Consensus | GQYHAQVIVTDQLVVGEPRRVSAKAVIKGNHLPVKLVSRFACFTLTAKYEMRLSCGHSTG |

| | | | |
|---|---|---|---|
| T3D | 661 | GQYHAQVIVTDQLVVGEPRRVSAKAVIKGNHLPVKLVSRFACFTLTAKYEMRLSCGHSTG | 720 |

| | | |
|---|---|---|
| T1L | 721 | RGAAYNARIAAFRSDLA 736 |

| | |
|---|---|
| Consensus | RGAAY ARLAFRSDLA |

| | | |
|---|---|---|
| T3D | 721 | RGAAYSARIIAFRSDLA 736 |

TABLE 5

Alignment of the nucleotide sequences of the T1L (GenBank Accession
No. X59945.1) and T3D (GenBank Accession No M27261.1) M1 cDNA encoding
mu-2 protein. The complete coding sequences are shown. Since reoviruses are
double-stranded RNA viruses, the reoviral genome would contain “u” in place to
“t”. Each genome segment shown below is 2304 nucleotides long that differ at 51
nucleotide positions.

| | | | |
|---|---|---|---|
| T1L | 1 | gctattcgcggtcatggcttacatcgcagttcctgcggtggtggattcacgttcaagtga | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1 | gctattcgcggtcatggcttacatcgcagttcctgcggtggtggattcacgttcgagtga | 60 |
| T1L | 61 | ggctattggactgctagaatcgtttggagtagacgctggggctgatgcgaatgacgtttc | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 61 | ggctattggactgctagaatcgtttggagtagacgctggggctgacgcgaatgacgtttc | 120 |
| T1L | 121 | atatcaagatcatgactatgtgttggatcagttacagtatatgttagatggatatgaggc | 180 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 121 | atatcaagatcatgactatgtgttggatcagttacagtacatgttagatggatatgaggc | 180 |
| T1L | 181 | tggcgacgttatcgatgcactcgtccacaagaattggttacatcactccgtctattgctt | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 181 | tggtgacgttatcgatgcactcgtccacaagaattggttacatcactctgtctattgctt | 240 |
| T1L | 241 | gttgccacccaaaagtcaactactagagtattggaaaagtaatccttcagtgataccgga | 300 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 241 | gttgccacccaaaagtcaactattagagtattggaaaagtaatccttcagcgataccgga | 300 |
| T1L | 301 | caacgttgatcgtcggcttcgtaaacgactaatgctaaagaaagatctcagaaaagatga | 360 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 301 | caacgttgatcgtcggcttcgtaaaegactaatgctaaagaaagatctcaggaaagatga | 360 |
| T1L | 361 | tgaatacaatcaactagcgcgtgctttcaagatatcggatgtctacgcacctctcatctc | 420 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 361 | tgaatacaatcagctagcgcgtgctttcaagatatcggatgtctacgcacctctcatctc | 420 |
| T1L | 421 | atccacgacgtcaccgatgacaatgatccagaacttgaatcaaggcgagatcgtgtacac | 480 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 421 | atccacgacgtcaccgatgacaatgatacagaacttgaatcgaggcgagatcgtgtacac | 480 |
| T1L | 481 | cacgacggacagggtaattggggctagaatcttgttatatgctcctagaaagtactatgc | 540 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 481 | cacgacggacagggtaataggggctagaatcttgttatatgctcctagaaagtactatgc | 540 |
| T1L | 541 | gtcaactctatcatttactatgactaagtgcatcattccgtttggcaaagaggtgggtcg | 600 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 541 | gtcaactctgtcatttactatgactaagtgcatcattccgtttggtaaagaggtgggtcg | 600 |
| T1L | 601 | tgttcctcactctagatttaatgttggcacatttccatcaattgctaccccgaaatgttt | 660 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 601 | tgttcctcactctcgatttaatgttggcacatttccgtcaattgctaccccgaaatgttt | 660 |
| T1L | 661 | tgtcatgagtggggttgatattgagtccatcccaaatgaattcatcaagttgttttacca | 720 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 661 | tgtcatgagtggggttgatattgagtccatcccaaatgaatttatcaagttgttttacca | 720 |
| T1L | 721 | gcgcgtcaagagtgttcacgccaatatactaaatgacatatcacctcagatcgtctctga | 780 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 721 | gcgcgtcaagagtgttcacgctaacatactaaatgacatatctcctcagatcgtctctga | 780 |
| T1L | 781 | catgataaacagaaagcgtttgcgcgttcatactccatcagatcgtcgagccgcgcagtt | 840 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 781 | catgataaacagaaagcgtctgcgcgttcatactccatcagatcgtcgagccgcgcagtt | 840 |
| T1L | 841 | gatgcatttgccctaccatgttaaacgaggagcgtctcacgtcgacgtttacaaggtgga | 900 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 841 | gatgcatttgccttaccatgttaaacgaggagcgtctcacgtcgacgtttacaaggtgga | 900 |
| T1L | 901 | tgttgtagacgtgttgttagaggtagtggatgtggccgatgggttgcgcaacgtatctag | 960 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 901 | tgttgtagacatgttgttcgaggtagtggatgtggccgatgggttgcgcaacgtatctag | 960 |
| T1L | 961 | gaaactaactatgcataccgttccggtatgtattcttgaaatgttgggtattgagattgc | 1020 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 961 | gaaactaactatgcataccgttcctgtatgtattcttgaaatgttgggtattgagattgc | 1020 |
| T1L | 1021 | ggactattgcattcgtcaagaggatggaatgttcacagattggttcctacttttaaccat | 1080 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1021 | ggactattgcattcgtcaagaggatggaatgctcacagattggttcctacttttaaccat | 1080 |
| T1L | 1081 | gctatctgatggcttaactgatagaaggacgcattgtcaatacttgattaatccgtcaag | 1140 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1081 | gctatctgatggcttgactgatagaaggacgcattgtcaatacttgatgaatccgtcaag | 1140 |
| T1L | 1141 | tgtgcctcctgatgtgatacttaacatctcaattactggatttataaataggcatacaat | 1200 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1141 | tgtgcctcctgatgtgatacttaacatctcaattactggatttataaatagacatacaat | 1200 |
| T1L | 1201 | cgatgtcatgcctgatatatatgacttcgttaaacccattggcgctgtgctgcctaaggg | 1260 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1201 | cgatgtcatgcctgacatatatgacttcgttaaacccattggcgctgtgctgcctaaggg | 1260 |
| T1L | 1261 | atcatttaaatcaacaattatgagagttcttgattcaatatcaatattaggagtccagat | 1320 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1261 | atcatttaaatcaacaattatgagagttcttgattcaatatcaatactaggaatccaaat | 1320 |
| T1L | 1321 | catgccgcgcgcgcatgtagttgactcagatgaggtgggcgagcaaatggagcctacgtt | 1380 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1321 | catgccgcgcgcgcatgtagttgactcagatgaggtgggcgagcaaatggagcctacgtt | 1380 |
| T1L | 1381 | tgagcatgcggttatggagatatacaaagggattgctggcgttgactcgctggatgatct | 1440 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1381 | tgagcaggcggttatggagatatacaaagggattgctggcgttgactcgctggatgatct | 1440 |
| T1L | 1441 | catcaagtgggtgctgaactcggatctcattccgcatgatgacaggcttggccaattatt | 1500 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1441 | catcaagtgggtgttgaactcggatctcattccgcatgatgacaggcttggtcaattatt | 1500 |
| T1L | 1501 | tcaagcgtttctgcctctcgcaaaggacttgttagctccaatggccagaaagttttatga | 1560 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1501 | tcaagcgtttttgcctctcgcaaaggacttattagctccaatggccagaaagttttatga | 1560 |
| T1L | 1561 | taactcaatgagtgagggtagattgctgacattcgctcatgccgacagtgagttgctgaa | 1620 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1561 | taactcaatgagtgagggtagattgctaacattcgctcatgccgacagtgagttgctgaa | 1620 |
| T1L | 1621 | cgcaaattactttggtcatttattgcgactaaaaataccatatattacagaggttaatct | 1680 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1621 | cgcaaattattttggtcatttattgcgactaaaaataccatatattacagaggttaatct | 1680 |
| T1L | 1681 | gatgattcgcaagaatcgtgagggtggagagctatttcagcttgtgttatcgtatctata | 1740 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1681 | gatgattcgcaagaatcgtgagggtggagagctatttcagcttgtgttatcttatctata | 1740 |
| T1L | 1741 | taaaatgtatgctactagcgcgcagcctaaatggtttggatcattattgcgattgttaat | 1800 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1741 | taaaatgtatgctactagcgcgcagcctaaatggtttggatcattattgcgattgttaat | 1800 |
| T1L | 1801 | atgtccctggttacatatggagaaattaataggagaagcagacccggcatctacgtcggc | 1860 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1801 | atgtccctggttacatatggagaaattaataggagaagcagacccggcatctacgtcggc | 1860 |
| T1L | 1861 | tgaaattggatggcatatccctcgtgaacagctgatgcaagatggatggtgtggatgtga | 1920 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1861 | tgaaattgggtggcatatccctcgtgaacagctgatgcaagatggatggtgtggatgtga | 1920 |
| T1L | 1921 | agatggattcattccctatgttagcatacgtgcgccaagactggttatggaggagttgat | 1980 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1921 | agacggattcattccctatgttagcatacgtgcgccaagactggttatagaggagttgat | 1980 |
| T1L | 1981 | ggagaagaactggggccaatatcatgcccaagttattgtcactgatcagcttgtcgtagg | 2040 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 1981 | ggagaagaactggggccaatatcatgcccaagttattgtcactgatcagcttgtcgtagg | 2040 |
| T1L | 2041 | cgaaccgcggagggtatctgccaaggctgtgatcaagggtaatcacttaccagttaagtt | 2100 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 2041 | cgaaccgcggagggtatctgctaaggctgtgatcaagggtaaccacttaccagttaagtt | 2100 |
| T1L | 2101 | agtttcacgatttgcatgtttcacattgacggcgaagtatgagatgaggctctcgtgcgg | 2160 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 2101 | agtttcacgatttgcatgtttcacattgacggcgaagtatgagatgaggctttcgtgcgg | 2160 |
| T1L | 2161 | ccatagcactggacggggggctgcatacaatgcgagactagctttccgatctgacttggc | 2220 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 2161 | ccatagcactggacgtggagctgcatacagtgcgagactagctttccgatctgacttggc | 2220 |
| T1L | 2221 | gtgatccgtgacatgcgtagtgtgacacctgcccctaggtcaatgggggtagggggcggg | 2280 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| T3D | 2221 | gtgatccgtgacatgcgtagtgtgacacctgctcctaggtcaatgggggtagggggcggg | 2280 |
| T1L | 2281 | ctaagactacgtacgcgcttcatc 2304 | |
| | | \|\|\|\|\|\|\|\|\|\|\|\|\|\|\|\|\|\|\|\|\|\|\|\| | |
| T3D | 2281 | ctaagactacgtacgcgcttcatc 2304 | |

Experiment 3: Assessment of Lethal Infection in PKR −/− vs. PKR +/+ Mice

Adult Balb-C PKR+/+ or PKR −/− mice were infected with various dosages of infectious reovirus T1L or T3D via the intraperitoneal (IP) or intranasal (IN) route. IP injections involved the administration of 0.1 ml of stock virus or virus diluted in PBS. IN infection involved the application of 0.05 ml volumes of stock virus or virus diluted in PBS onto the nose-pad of mice anaesthetized with halothane (administered at 3% in oxygen). The survival of adult mice was monitored over a 30 day period. Adult PKR +/+ and PKR −/− mice resisted infection with 5e6 infectious T3D virus whereas T1L virus killed PKR −/− mice but not PKR +/+ mice at this dose. This demonstrates an enhanced ability of T1L to infect the tissues of PKR −/− mice. Table 5.

Two day old suckling Balb-C PKR +/+ or PKR −/− mice were infected with various dosages of infectious reovirus T1L or T3D via the IN route. IN infectious involved the application of 0.01 ml volumes of stock virus or virus diluted in PBS onto the nose-pad of mice anaesthetized with halothane (administered at 3% in oxygen). The survival of suckling mice was monitored over an 18 day period. Suckling PKR +/+ or PKR −/− mice were both susceptible to similar dosages of T1L whereas T3D virus killed PKR −/− mice much more effectively than PKR +/+ mice, killing them at doses more than 100 fold less than those required to kill wild type suckling mice. This demonstrates an enhanced ability of T3D to infect the tissues of PKR −/− tissues of suckling mice and indicates a difference in the properties of the T1L and the T3D strains with respect to differential replication in PKR +/+ versus PKR −/− mice although both viruses were more restricted in replication of PKR +/+ mice of different ages (adult versus suckling). Table 5.

TABLE 5

| | T1L virus (S/So) | | T3D virus (S/So) | |
|---|---|---|---|---|
| | PKR+/+ | PKR−/− | PKR+/+ | PKR−/− |
| ADULT | | | | |
| MICE | | | | |
| 5 E6 IP | ND | 100% (3/3) | ND | 100% (3/3) |
| 5 E6 IN | 100% (3/3) | 0% (0/3) | 100% (3/3) | 100% (3/3) |
| 5 E5 IN | ND | 100% (3/3) | ND | ND |
| SUCKLING | | | | |
| MICE | | | | |
| 3 E6 IN | 33% (2/6) | 66% (2/3) | 84% (5/6) | 0% (0/2) |
| 3 E4 IN | 100% (7/7) | ND | 100% (7/7) | 0% (0/4) |
| 3 E3 IN | ND | ND | ND | 100% (3/3) |

Experiment 4: Reovirus T3D is a Stronger Inducer of PKR MEF than T1L

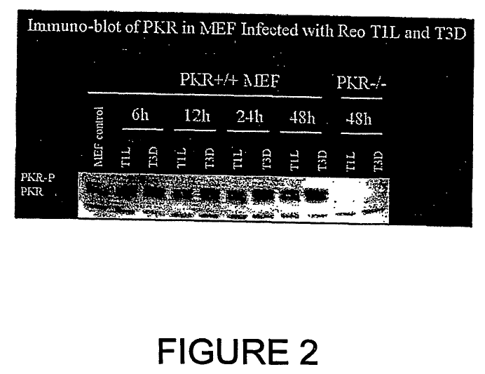

Infection of PKR +/+ MEF results in a greater expression of the phosphorylated form of PKR (FIG. 2). PKR +/+ MEF were infected at a moi of 10 and incubated over a 48 hr period for immunoblot analysis using rabbit anti-PKR serum that reacts with the first 100 amino acids of PKR. Proteins were separated on a 10% polyacrylamide gel and transferred to IMMOBILON membrane (Millipore Inc.) before incubation with 1/100 diluted primary antibody in the presence of casein. After repeated washing the blot was incubated with goat anti-rabbit antibody conjugated with alkaline phospatase (1/30,000 dilution) (Sigma Inc) for 1 hour before repeated washing and reaction with Attophos substrate for phosphorescent detection as shown in FIG. 2. Activation of PKR results in an electrophoretic form of slightly slower mobility indicated as PKR-P. Infection with T3D results in a greater production of this form than with infection with T1L. This demonstrates that PKR expression is enhanced in T3D infected cells and indicates that this may be responsible for the greater sensitivity of this virus to the PKR gene.

Experiment 5: Proof of Principle for Improved Oncolysis of Reovirus T1L×T3D Reassortants: Demonstration that Reovirus Reassortants with the M1 Gene of T3D and the Remaining Genes from T1L and T3D have Superior Oncolytic Properties.

Three reassortants were chosen for testing of oncolytic properties relative to their parental viruses. Each of the reassortants, EB96, EB108 and EB146 posessed the M1 gene of T3D and were expected to preferentially replicate in cells that were damaged in their interferon response. These reassortants also possessed their L1, L3 and S2 genes of T1L that would be predicted to provide optimal replication abilities.

Oncolytic testing was performed by intranasal infection of 10⁷ pfu of each virus into mice that possessed lung tumors derived form the CT26 colon tumor cell line fo Balb-C origin. Adult female BALB-C mice, 4-6 weeks old, were injected in the tail vein with 3×10⁵ CT 26 on day 0 of the experiment. On day 7 groups of 3 mice were anaesthetized and infected with 10⁷ pfu of virus in a 0.050 volume of culture medium. Mice were housed for an additional 6 days before euthanization with 90% CO₂/10% O₂. Lungs were removed, weighed, fixed in formalin and photographed. One set of lungs was examined histopathologically by hematoxylin and eosin staining after paraffin embedding and sectioning.

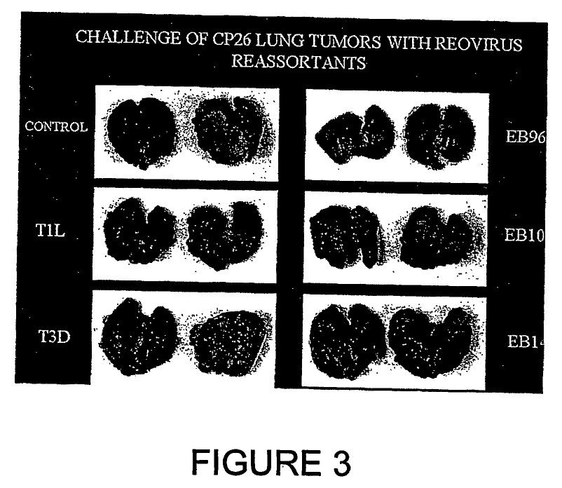

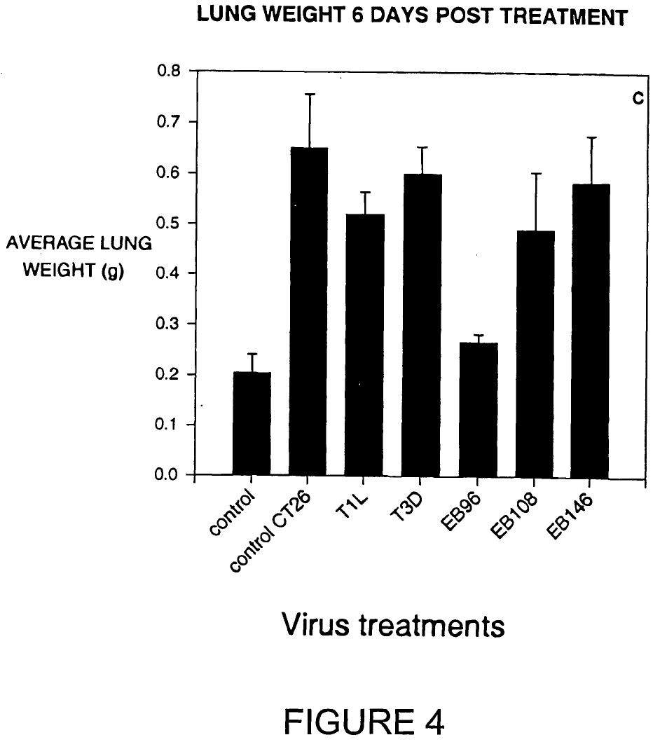

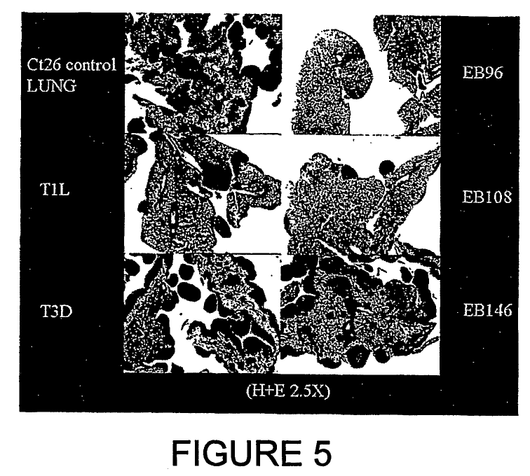

The gross appearance of lungs after treatment showed that the untreated control lungs were heavily tumor laden having a pebbled surface appearance due to contiguous tumor nodules (FIG. 3). These animals were in the terminal stages of cancer since one animal died at this time and the others were in respiratory distress. These lungs were 3 times heavier than uninfected balb-c lungs indicating the increased tumor mass approximated twice the mass of the lung tissue (FIG. 4). Histologically these lungs were covered with a contiguous layer of tumor nodules and internal tumor masses seen as eosinophilic growths of cells (FIGS. 4 and 5). Infection with T1L virus resulted in a partial freeing of surface tumor growth observable on gross inspection that was also associated with a decrease in interior and surface nodules and a 20% reduction in lung weight relative to untreated control (FIG. 3, 4 and 5). T3D treatment was not as effective as T1L resulting in lungs that were only distinguishable form untreated controls by a slight (8%) decrease in size but were similar in gross and microsopic appearance of tumors (FIG. 3, 4 and 5).

In dramatic contrast the EB96 reassortant virus cleared the lung of gross tumor mass on treatment (FIG. 3). The lungs were of approximately normal weight having been freed of tumor masses (FIG. 4). A small number of residual tumor cells remained at this time as detected by histological examination (FIG. 5). The lungs were of normal size and appearance except for some circular patterns and dents on the lungs surface that presumably marked the location of prior tumor nodules. EB146 virus was not more effective at tumor lysis than the T3D parental virus (FIG. 3, 4 and 5). Reassortant EB108 was partially effective at oncolysis producing results that were marginally better but similar than the T1L parental strain. On comparison of the genotyoes of the reassortants it can be seen that the 3 ressortants possess 7 genome segments in common and thus differ in their L2, S3 and S4 genome segments indicating that the latter group of genes include important modulators of oncolysis. The EB96 reassortant is more effective than EB108 soley due to the nature of the S4 gene since these viruses only differ in the parental origin of this gene. This indicates that the T1L S4 gene conferred enhanced oncolytic properties relative to the T3D S4 gene. Since the S4 gene encodes the dsRNA binding protein that blocks PKR activation it is possible that the T1L S4 gene differs in this ability and thus, in concert with other combinations of T1L and T3D genome segments, controls oncolytic potential. In conclusion, the dramatic increase in effectiveness of the EB96 reassortant at oncolysis, relative to the parental T1L and T3D viruses demonstrates the proof of principle that reassortants of reovirus with specific genotyoes have enhanced and effective tumor lysis abilities in metastatic tumors in hosts with active immune responses. Table 6.

TABLE 6
Ranking of the ability of reovirus reassortants to lyse ct26 lung
tumors. The relative weight of ct26 tumor bearing lungs relative to
untreated control tumor bearing lungs are shown. The parental origin
of genome segments are indicated as L for T1L and D for T3D.

| | TU- | | | | | | | | | | | |
|---|---|---|---|---|---|---|---|---|---|---|---|---|
| | MOR | | | | | | | | | | | |
| VIRUS | % | L1 | L2 | L3 | M1 | M2 | M3 | S1 | S2 | S3 | S4 | RANK |
| eb96 | 41 | L | D | L | D | L | L | L | L | D | L | 1 |
| eb108 | 75 | L | D | L | D | L | L | L | L | D | D | 2 |
| T1L | 80 | L | L | L | L | L | L | L | L | L | L | 3 |
| eb146 | 89 | L | L | L | D | L | L | L | L | L | D | 4 |
| T3D | 92 | D | D | D | D | D | D | D | D | D | D | 5 |

Experiment 6: Ability of T1L×T3D Reassortants to Lyse Tumors In Vitro

A panel of tumor cell lines obtained fron the NCI tumor panel (SF539, cns; SKMEL28, melanoma; HT29; NCI H123, nsc-lung; SW620, colon; DU145, prostate) were infected with the T1L, T3D, or the reassortants, EB96, EB108 and. EB146 at an moi of 10 and were observed for cytopathic effect over a 5 day period. The ability to lyse tumor cells was scored visually on a scale of − to +++, where − indicates no difference form mock infected cells and +, ++, and +++ indicate 33% cell destruction, 66% cell destruction and complete lysis respectively. Although different tumor cell types differed in their susceptibility to lysis by different reovirus parents or reassortants the reassortants viruses were all as effective or more effective than the T3D parental virus at tumor cell lysis in vitro (Table 7).

TABLE 7
Cytopathology of reovirus T1L and T3D and reassortants in
different tumor cell lines

| | Tumor cell line | | | | | |
|---|---|---|---|---|---|---|
| | SF539 | SKMEL28 | HT29 | NCI H23 | SW620 | DU145 |
| virus | Cns | melanoma | − | nsc-lung | colon | prostate |
| T1L | ++ | +++ | ++ | +++ | − | ++ |
| T3D | − | +++ | + | ++ | − | + |
| EB96 | ++ | +++ | ++ | ++ | + | + |
| EB108 | ++ | +++ | ++ | ++ | + | + |
| EB146 | ++ | +++ | ++ | +++ | + | ++ |
| RAS | | | | | | |

What is claimed is:

1. A method of reducing the viability of a tumor cell, comprising administering to the tumor cell a non-naturally occurring virus wherein the virus is:
a) a reovirus whose mu-2 protein has amino acid residues A, R, M, F, L, M, I, Q, I and S at positions 93, 150, 300, 302, 347, 372, 434, 458, 652 and 726, respectively; or
b) a reassortant of two or more parent strains of a viral species selected from the family Reoviridae, or progeny thereof, or
c) a virus other than a reovirus capable of expressing a reovirus mu-2 protein having amino acid residues A, R, M, F, L, M, I, Q, I and S at positions 93, 150, 300, 302, 347, 372, 434, 458, 652 and 726, respectively, wherein the virus other than a reovirus is a DNA virus, a positive-sense RNA virus, or a negative-sense RNA virus selected from the group consisting of Orthomyxoviridae, Rhabdoviridae and Paramyxoviridae.

2. A method of infecting a neoplasm in a mammal with a virus, comprising administering to the mammal a non-naturally virus wherein the virus is:
a) a reovirus whose mu-2 protein has amino acid residues A, R, M, F, L, M, I, Q, I and S at positions 93, 150, 300, 302, 347, 372, 434, 458, 652 and 726, respectively; or
b) a reassortant of two or more parent stains of a viral species selected from the family Reoviridae, or progeny thereof; or
c) a virus other than a reovirus wherein the virus other than a reovirus is:
i) capable of expressing a reovirus mu-2 protein having amino acid residues A, R, M, F, L, M, I, Q, I and S at positions 93, 150, 300, 302, 347, 372, 434, 458, 652 and 726, respectively, and
ii) is a DNA virus, a positive-sense RNA virus, or a negative-sense RNA virus selected from the group consisting of Orthomyxoviridae, Rhabdoviridae and Paramyxoviridae.

3. A method of treating a neoplasm in a mammal comprising administering to the mammal a therapeutically effective amount of a non-naturally occurring virus wherein the virus is:
a) a reovirus whose mu-2 protein has amino acid residues A, R, M, F, L, M, I, Q, I and S at positions 93, 150, 300, 302, 347, 372, 434, 458, 652 and 726, respectively; or
b) a reassortant of two or more parent strains of a viral species selected from the family Reoviridae, or progeny thereof; or
c) a virus other than a reovirus wherein the virus other than a reovirus is:
i) capable of expressing a reovirus mu-2 protein having amino acid residues A, R, M, F, L, M, I, Q, I and S at positions 93, 150, 300, 302, 347, 372, 434, 458, 652 and 726, respectively, and
ii) is a DNA virus, a positive-sense RNA virus, or a negative-sense RNA virus selected from the group consisting of Orthomyxoviridae, Rhabdoviridae and Paramyxoviridae.

4. Use of a non-naturally occurring virus in the manufacture of a medicament for reducing the viability of a tumor cell, infecting a neoplasm in a mammal, or treating a neoplasm in a mammal, wherein the virus is:
a) a reovirus whose mu-2 protein has amino acid residues A, R, M, F, L, M, I, Q, I and S at positions 93, 150, 300, 302, 347, 372, 434, 458, 652 and 726, respectively; or
b) a reassortant of two or more parent strains of a viral species selected from the family Reoviridae, or progeny thereof; or
c) a virus other than a reovirus wherein the virus other than a reovirus is:
i) capable of expressing a reovirus mu-2 protein having amino acid residues A, R, M, F, L, M, I, Q, I and S at positions 93, 150, 300, 302, 347, 372, 434, 458, 652 and 726, respectively, and
ii) is a DNA virus, a positive-sense RNA virus, or a negative-sense RNA virus selected from the group consisting of Orthomyxoviridae, Rhabdoviridae and Paramyxoviridae.

5. The method of claim 1, 2 or 3, or the use of claim 4, wherein the virus is a reovirus whose mu-2 protein has amino acid residues A, R, M, F, L, M, I, Q, I and S at positions 93, 150, 300, 302, 347, 372, 434, 458, 652 and 726, respectively.

6. The method or use of claim 5, wherein the mu-2 protein has the amino acid sequence of the mu-2 protein of reovirus strain T3 Dearing.

7. The method or use of claim 6, wherein the mu-2 protein is expressed by a gene having the nucleic acid sequence of the M1 gene of reovirus strain T3 Dearing.

8. The method of claim 7, wherein the reovirus has the same genotype as a reovirus strain selected from the group consisting of eb86, eb129, eb88, eb13, and eb145.

9. The method or use of claim 7, wherein the reovirus has a L3 gene whose sequence is the same as the L3 gene of reovirus strain T1 Lang.

10. The method or use of claim 9, wherein the reovirus has the same genotype as a reovirus strain selected from the group consisting of eb28, eb31, eb97, eb123 and g16.

11. The method of claim 9, wherein the reovirus has a L1 gene and a S2 gene whose sequences are the same as the corresponding genes of reovirus strain T1 Lang.

12. The method of claim 11, wherein the reovirus has the same genotype as a reovirus strain selected from eb146 and eb108.

13. The method of claim 11, wherein the reovirus has a S4 gene whose sequence is the same as the corresponding gene of reovirus strain T1 Lang.

14. The method of claim 12, wherein the reovirus has the same genotype as reovirus strain eb96.

15. The method of claim 1, 2 or 3 or the use of claim 4, wherein the virus is a reassortant of two or more parent strains of a viral species selected from the family Reoviridae, or progeny thereof.

16. The method or use of claim 15, wherein the viral species is reovirus and the parent strains are selected from the group consisting of T3 Dearing, T1 Lang, T3 Abney, and T2 Jones.

17. The method or use of claim 16, wherein the parent strains are T3 Dearing and T1 Lang.

18. The method or use of claim 17, wherein the virus is selected from the group consisting of viral strains eb118, eb73.1, h17, h15, eb39, and h60.

19. The method of claim 1, 2 or 3 or the use of claim 4, wherein the virus is a virus other than a reovirus wherein the virus other than a reovirus is:
i) capable of expressing a reovirus mu-2 protein having amino acid residues A, R, M, F, L, M, I, Q, I and S at positions 93, 150, 300, 302, 347, 372, 434, 458, 652 and 726, respectively, and
ii) is a DNA virus, a positive-sense RNA virus, or a negative-sense RNA virus selected from the group consisting of Orthomyxoviridae, Rhabdoviridae and Paramyxoviridae.

20. The method or use of claim 19, wherein the virus is a DNA virus selected from a Herpesvirus, Adenovirus, Parvovirus, Papovavirus, Iridovirus, Hepadenavirus, Poxvirus, mumps virus, human parainfluenza virus, measles virus or rubella virus.

21. The method or use of claim 19, wherein the virus is a positive-sense RNA virus selected from a Togavirus, Flavivirus, Picomavirus, or Coronavirus.

22. The method or use of claim 19, wherein the virus is a negative-sense RNA virus selected from the group consisting of Orthomyxoviridae, Rhabdoviridae and Paramyxoviridae.

23. The method or use of claim 19, wherein the virus is an influenza virus or a vesicular stomatitis virus.

24. The method or use of any one of claims 1-23, wherein the virus is a replication competent virus.

25. The method or use of claim 24, wherein the virus is a clonal virus.

26. The method of any one of claims 1-25, wherein the virus is administered by a route selected from the group consisting of intranasally, intratracheally, intravenously, intraperitoneally or intratumorally.

27. The method or use of any one of claims 1-26 wherein the virus is administered to a human or non-human mammal.

28. The method or use of claim 26 or 27 wherein the virus is administered at a dose of from 3×10⁷ to 3×10⁹ PFU/kg.
